(19) United States
(12) Patent Application Publication
(10) Pub. No.: US 2020/0255785 A1
(43) Pub. Date: Aug. 13, 2020

(54) ONLINE BIOMASS CAPACITANCE MONITORING DURING LARGE SCALE PRODUCTION OF POLYPEPTIDES OF INTEREST

(71) Applicant: Bristol-Myers Squibb Company, Princeton, NJ (US)

(72) Inventors: Timothy C. ERLANDSON, Berlin, MA (US); Joon Chong YEE, Acton, MA (US); Sarbajita RAY, Boxborough, MA (US); Michael Christopher BOYS, Groton, MA (US); Charles Anthony HILL, Groton, MA (US)

(73) Assignee: Bristol-Myers Squibb Company, Princeton, NJ (US)

(21) Appl. No.: 16/647,038

(22) PCT Filed: Sep. 14, 2018
(86) PCT No.: PCT/US2018/051100

§ 371 Date: Mar. 13, 2020

Related U.S. Application Data
(60) Provisional application No. 62/559,142, filed on Sep. 15, 2017.

(51) Int. Cl. C12M 1/00; C12M 1/34

(52) U.S. Cl. CPC C12M 29/10; C12M 41/46

(57) ABSTRACT

The present invention relates to the use of online biomass capacitance monitoring in perfusion cultures as a way to minimize volumes of cell culture media in large-scale manufacturing. In certain embodiments, a biomass capacitance probe is used to measure the viable cell density, and the viable cell density is used in conjunction with a pre-determined constant cell-specific perfusion rate to identify a target perfusion rate.

### CROSS-REFERENCE TO RELATED APPLICATIONS

This application claims priority to U.S. Provisional Application No. 62/559,142, filed Sep. 15, 2017, which is incorporated herein by reference in its entirety.

### BACKGROUND OF THE INVENTION

A perfusion culture utilizes a cell separation device or filtration methods to retain cells in a bioreactor, while exchanging out spent media with fresh media. Perfusion rates are typically set between one to five bioreactor volumes per day, and maintained constant on a 24 hour basis. Perfusion rates can be increased based on viable cell density measurements to ensure adequate nutrients for the cells. Due to the fact that this perfusion rate is only adjusted on an infrequent, daily basis, the amount of media exchanged can be either under or over-estimated on a per cell basis. Under estimation of an actual cell-specific perfusion rate could result in depletion of nutrients and thus impact cell growth rates. An over-estimation of cell-specific perfusion rate results in high media perfusion rates without fully utilizing the nutrients provided in the media.

Others have measured viable cell density and the perfusion rate during the production phase. Dowd et al., Cytotechnology 42: 35-45 (2003). However, there is a need in the art for mechanisms to dynamically adjust perfusion rates to meet a target rate, thereby reducing media usage while retaining a constant level of nutrients in a high cell density perfusion system.

### SUMMARY OF THE INVENTION

In one embodiment, the present invention relates to a method of controlling the perfusion rate in a perfusion bioreactor comprising: a) measuring the viable cell density of cells growing in cuture using a biomass capacitance probe; and b) dynamically adjusting the target perfusion rate in view of the viable cell density, wherein the cells are grown to a high cell density during perfusion.

In one embodiment, the present invention relates to a method of minimizing cell culture medium perfusion volume comprising: a) measuring the viable cell density of cells growing in cuture using a biomass capacitance probe; and b) dynamically adjusting the target perfusion rate in view of the viable cell density, wherein the cells are grown to a high cell density during perfusion.

In some embodiments, the viable cell density is used in combination with a constant cell-specific perfusion rate to determine the target perfusion rate. In certain embodiments, the constant cell-specific perfusion rate is between about 0.01 to 0.15 nL/cell/day. In particular embodiments, the constant cell-specific perfusion rate is between about 0.02 to 0.10 nL/cell/day. In one embodiment, the constant cell-specific perfusion rate is about 0.04 nl/cell/day. In embodiments, the constant cell-specific perfusion rate is about 0.03 nl/cell/day. In some embodiments, the constant cell-specific perfusion rate is about 0.04-0.05 nl/cell/day. In certain embodiments, the constant cell-specific perfusion rate is about 0.04 nl/cell/day. In particular embodiments, the constant cell-specific perfusion rate is between about 0.08 to 0.10 nL/cell/day. In embodiments, the constant cell-specific perfusion rate is about 0.10 nl/cell/day

In some embodiments, the calculation of the target perfusion flow rate is performed by a bioreactor controller. In certain embodiments, the perfusion flow rate is increased to achieve the target perfusion flow rate following measurement of the viable cell density in the bioreactor. In other embodiments, the perfusion flow rate is decreased to achieve the target perfusion flow rate following measurement of the viable cell density in the bioreactor.

In some embodiments, the viable cell density is further used to measure the time point when a target cell density is reached. In certain embodiments, the target cell density is >30×106 cells/ml, >40×10⁶ cells/ml, >50×10⁶ cells/ml, >60×10⁶ cells/ml, or >70×10⁶ cells/ml. In particular embodiments, the target cell density is >40×10⁶ cells/ml.

In some embodiments, the total perfusate is measured. In embodiments, the daily perfusion rate is measured. In certain embodiments, the capacitance is measured.

In embodiments, the cells are transferred into a production bioreactor upon reaching the target cell density. In certain embodiments, the cells produce a polypeptide of interest. In particular embodiments, the polypeptide of interest is an antibody.

In embodiments, the cells are mammalian cells. In certain embodiments, the cells are CHO cells.

In some embodiments, the perfusion bioreactor is an N-1 perfusion bioreactor. In embodiments, the perfusion bioreactor is attached to an alternating tangential flow system.

In embodiments, the perfusion bioreactor uses less than about 5, less than about 4.5, less than about 4, less than about 3.5, less than about 3.0, less than about 2.5, less than about 2, less than about 1.5 or less than 1 vessel volumes of perfusion media per day. In particular embodiments, the perfusion bioreactor uses only about 25%, about 30%, about 35%, about 40%, about 45%, about 50%, about 55%, about 60%, about 65%, about 70%, about 75%, about 80%, about 85%, about 90%, or about 95% of the total amount of perfusion media used by a process that does not measure the viable cell density.

In embodiments, the biomass capacitance probe is an INCYTE probe.

### BRIEF DESCRIPTION OF THE DRAWINGS

FIG. 1 : Schematic diagram for the control of perfusion rate in a 5-L bioreactor with ATF 2 perfusion device and biomass capacitance probe.

FIG. 2: Schematic diagram for the control of perfusion rate in a 200-L bioreactor with ATF 6 perfusion device and biomass capacitance probe.

FIGS. 3A and 3B: An online estimation of viable cell density based on a linear fit of capacitance reading to the offline viable cell densities. Data points are marked according to different bioreactor runs. The equation that describes correlation of capacitance reading to viable cell density profiles is shown for two recombinant CHO cell lines in (A) and (B). Seven total bioreactor runs from cell line A were used to generate data in plot A and two runs from cell line B were used to generate data in plot B.

FIGS. 4A and 4B: (A) Media utilization rates over time using two different modes of perfusion. Diamond: perfusion rate adjusted dynamically based on online VCD readings from bio-capacitance probe. Circle: perfusion rate adjusted every 24 hours based on offline VCD measurements. (B) Cell density profiles for the 5-L and 200-L perfusion bioreactors. Cell growth rates are equivalent independent of the method for controlling perfusion rate.

FIGS. 5A and 5B: (A) High and low cell specific perfusion rates at the N-1 seed bioreactor resulted in differences in viable cell density profiles for cell line C. At 144 hours and 216 hours a portion of the cells were inoculated into production bioreactor and the cells were re-seeded in the current vessel for continuing cell growth. (B) Glucose levels in the N-1 bioreactor at two different perfusion rates. The lower perfusion rates resulted in depletion of glucose at 144 hours.

FIGS. 6A and 6B: Effect of perfusion rate on viable cell density. Perfusion N-1 seed bioreactors were used to inoculate production vessels at either 6 or 10×10⁶ cells/ml. The culture that is inoculated from a N-1 seed operated at 0.04 nL/cell/day yield a higher peak VCD (square and triangle lines) compared to N-1 seed operated at 0.02 nL/cell/day.

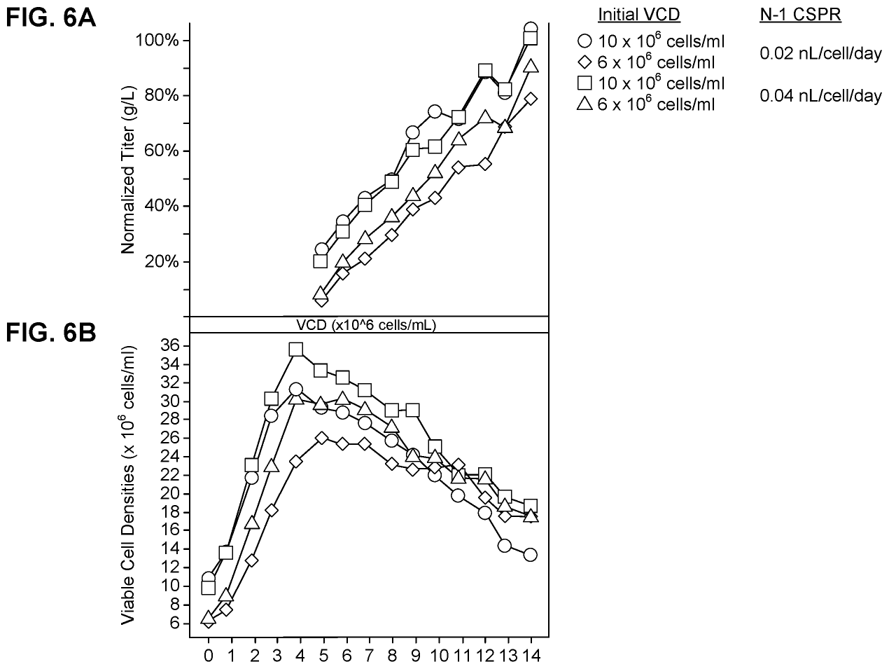

FIG. 7A-7F: Dynamic adjustment of perfusion rate based on online capacitance values demonstrated in cell-line D in both 5-L and 200-L scale. The target CSPR for this cell line is set at 0.10 nL/cell-day. (A) viable cell densities, capacitance (B) total perfusate, daily perfusion rate, (C) cell-specific perfusion rate in 200-L scale. (D) viable cell densities, capacitance (E) total perfusate, daily perfusion rate; (F) cell-specific perfusion rate in 5-L scale.

FIG. 8A-8C: Dynamic adjustment of perfusion rate based on online capacitance values demonstrated in cell-line C in 5-L scale. The target CSPR for this cell line is set at 0.04 nL/cell-day. (A) viable cell densities, capacitance (B) total perfusate, daily perfusion rate, (C) cell-specific perfusion rate in 5-L scale.

FIG. 9A-9C: Dynamic adjustment of perfusion rate based on online capacitance values demonstrated in cell-line E in 5-L scale. The target CSPR for this cell line is set at 0.04 nL/cell-day. (A) viable cell densities, capacitance (B) total perfusate, daily perfusion rate, (C) cell-specific perfusion rate in 5-L scale.

FIG. 10:A-10F Dynamic adjustment of perfusion rate based on online capacitance values demonstrated in cell-line A in 5-L scale. (A) VCD and capacitance, (B) total perfusate volume and daily perfusion rate, (C) cell specific perfusion rate at target of 0.03 nL/cell-day. (D) VCD and capacitance, (E) total perfusate volume and daily perfusion rate, (F) cell specific perfusion rate at target of 0.04 nL/cell-day.

### DETAILED DESCRIPTION OF THE INVENTION

### Definitions of General Terms and Expressions

In order that the present disclosure may be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of can mean a range of up to 10% or 20% (i.e., ±10% or ±20%). For example, about 3mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “cell culture” and “culture” include any combination of cells and medium. The methods of the present invention contemplate, without limitation, perfusion cell culture and fed-batch cell culture.

As used herein, the terms “perfuse”, “perfusion” and “perfusion culture” are used interchangeably to refer to a method of culturing cells, wherein additional fresh medium is provided to the culture and spent medium is removed from the culture. Perfusion is initiated after the culture is seeded and can occur either continuously or intermittently, as desired, over a period of time. The fresh medium added during perfusion typically provides nutritional supplements for the cells that have been depleted during the culturing process. Perfusion also allows for removal of cellular waste products and toxic byproducts from the cell culture. Perfusion is performed during the growth phase of the cells, but can also be continued after the cells have been transferred to a fed-batch cell culture.

As used herein, the term “VVD” is a value set by the perfusion rate and refers to the volume vessel volume exchanges per day.

As used herein, the term “specific perfusion rate” (SPR) refers to the rate at which fresh medium is provided to the cell culture and spent medium is removed based on cell density. In embodiments, the SPR is a constant cell-specific perfusion rate (CSPR). In embodiments, the CSPR equals the perfusion rate/cell density. In certain embodiments, the SPR is used to calculate the target perfusion rate or the target flow rate, described in Equation 1. In embodiments, the target perfusion rate is the amount of media that is pumped out of the bioreactor.

The SPR can be determined and/or adjusted by one of ordinary skill in the art, based on factors including, but not limited to the nature of the particular host cells, the cell growth rate, and the cell density, including the viable cell density. For example, the typical SPR can range from about 0.01 to about 1.0 nL/cell/day/. In one embodiment, the SPR is about 1.0, about 0.9, about 0.8, about 0.7, about 0.6, about 0.5, about 0.4, about 0.3, about 0.2, about 0.1, about 0.09, about 0.08, about 0.07, about 0.06, about 0.05, about 0.04, about 0.03, about 0.02 or about 0.01 nL/cell/day. In embodiments, the SPR is about 0.05 nL/cell/day. In a particular embodiments, the SPR is about 0.04 nL/cell/day. In other embodiments, the SPR is about 0.03 nL/cell/day. In yet other embodiments, the SPR is about 0.02 nL/cell/day. In some embodiments, the SPR is about 0.10 nL/cell/day. In embodiments, the SPR is about 0.09 nL/cell/day. In embodiments, the SPR is about 0.08 nL/cell/day. In certain embodiments, the SPR is between about 0.01 to about 0.2 nL/cell/day, about 0.01 to about 0.15 nL/cell/day, about 0.02 to about 0.10 nL/cell/day, about 0.02 to about 0.05 nl/cell/day, about 0.03 to about 0.05 nl/cell/day, or about 0.04 to about 0.05 nl/cell/day . In other embodiments, the SPR is about 0.05 to about 0.15 nl/cell/day, about 0.06 to about 0.15 nl/cell/day, 0.07 to about 0.15 nl/cell/day, 0.08 to about 0.15 nl/cell/day, 0.05 to about 0.14 nl/cell/day, 0.05 to about 0.13 nl/cell/day, 0.05 to about 0.12 nl/cell/day, 0.05 to about 0.11 nl/cell/day, 0.05 to about 0.10 nl/cell/day, 0.06 to about 0.12 nl/cell/day, or 0.08 to about 0.10 nl/cell/day,

The SPR can remain constant over a period of time or it can be altered (i.e., increased or decreased) over the course of the period of perfusion. For example, the SPR can be steadily increased over time, increased in a step-wise fashion over time, steadily decreased over time, decreased in a step-wise fashion over time, or any combination thereof. In certain embodiments, the SPR is adjusted following the measurement of the viable cell density. The perfusion can be applied in a continuous manner or in an intermittent manner. One of ordinary skill in the art can determine the SPR, as well as the appropriate timing of the initiation and cessation of the perfusion period(s), and of any alterations to the perfusion, based upon the monitoring of some parameter of the cell culture.

As used herein, the term “fed-batch culture” refers to a method of culturing cells, wherein the cell culture is supplemented with fresh medium, i.e., the cells are “fed” with new medium while spent medium is not removed. Typically, a “fed-batch” culture process is performed in a bioreactor and additional components (e.g., nutritional supplements) are added to the culture at some time after initiation of the culture process. The controlled addition of nutrients directly affects the growth rate of the culture and allows for avoidance of the build-up of overflow metabolites (see, for example, Wlaschin, K. F. et al., “Fedbatch culture and dynamic nutrient feeding,” Cell Culture Engineering, 101:43-74 (2006) and Lee, J. et al., “Control of fed-batch fermentations,” Biotechnol. Adv., 17:29-48 (1999)). A fed-batch culture is typically terminated at some point and the cells and/or components in the medium are harvested and optionally purified.

As used herein, the terms “inoculation”, “inoculum”, and “seeding” refer to the addition of cells to starting medium to begin the culture.

As used herein, the term “cell density” refers to the number of cells in a given volume of medium. Cell density can be monitored by any technique known in the art, including, but not limited to, extracting samples from a culture and analyzing the cells under a microscope, using a commercially available cell counting device or by using a commercially available suitable probe introduced into the bioreactor itself (or into a loop through which the medium and suspended cells are passed and then returned to the bioreactor). In embodiments, a biomass capacitance probe measures capacitance, which is correlated to cell density.

As used herein the terms “super high cell density” and “high cell density” are used interchangeably and refer to a cell density of at least about 40×10⁶ cells/mL in an N-1 perfusion bioreactor. Known cell culture techniques may involve growing cells to a “first critical level” (i.e., a point during the cell cycle growth phase when the cell viability may be affected by the increased concentration of waste productions (e.g., cell growth inhibitors and toxic metabolites, e.g., lactate, ammonium, etc.) before perfusing the cell culture and obtaining roughly 5 to 40 million cells/mL). In contrast, cells grown according to the methods of the present invention are able to reach a high cell density. In some embodiments, cells of the present invention are grown to target cell densities of least above about 40, 45, 50, 55, 60, 65, 70, 75, 80, 90, 100, 110, 120, or 130×10⁶ cells/mL. In particular embodiments, cells of the present invention are grown to target cell densities of about 60×10⁶ cells/mL. In other embodiments, cells of the present invention are grown to target cell densities of about 40×10⁶ cells/mL. High density seeding refers to inoculating cultures at about 5×10⁶ cells/ml, about 10×10⁶ cells/ml, about 15×10⁶ cells/ml, about 20×10⁶ cells/ml, or about 25×10⁶ cells/ml. In certain embodiments, high density seeding refers to inoculating cultures at about 10×10⁶ cells/ml.

As used herein, the term “viable cell density” or “VCD” refers to the number of live cells present in a given volume of medium under a given set of experimental conditions.

As used herein, the term “cell viability” refers to the ability of cells in culture to survive under a given set of conditions or experimental variations. The term as used herein also refers to that portion of cells that are alive at a particular time in relation to the total number of cells (e.g., living and dead) in the culture at that time.

As used herein, the “growth phase” of a cell culture refers to the phase during which the viable cell density at any time point is higher than at any previous time point.

As used herein, the “production phase” of a cell culture refers to the phase during which the cells produce significant amounts of protein, which accumulates for future processing.

As used herein, the term “cell integral” refers to the overall viable cell numbers during the course of a cell growth profile.

As used herein, the term “titer” refers to the total amount of protein produced by a cell culture, divided by a given amount of medium volume. In essence, the term “titer” refers to a concentration and is typically expressed in units of milligrams of polypeptide per liter of medium. The methods of the present invention have the effect of substantially increasing polypeptide product titer, as compared to polypeptide product titer produced from other cell culture methods known in the art.

As used herein, the terms “media”, “cell culture media” and “culture media”, including grammatical variations thereof, are used interchangeably, and refer to the nutrient solution in which cells (for example, animal or mammalian cells) are grown in culture. Cell culture media is the physiochemical, nutritional, and hormonal environment for cells and typically includes at least one or more components from the following: an energy source (e.g., in the form of a carbohydrate such as glucose); essential amino acids, including the twenty basic amino acids plus cysteine; vitamins and/or other organic compounds typically required at low concentrations; lipids or free fatty acids (e.g., linoleic acid); and trace elements (e.g., inorganic compounds or naturally occurring elements that are typically required at very low concentrations, usually in the micromolar range). Media may be solid, gelatinous, liquid, gaseous or a mixture of phases and materials.

As used herein, the term “cell”, refers to animal cells, mammalian cells, cultured cells, host cells, recombinant cells, and recombinant host cells. Such cells are generally cell lines obtained or derived from mammalian tissues which are able to grow and survive when placed in media containing appropriate nutrients and/or growth factors. The cells utilized in the methods of the present invention are generally animal or mammalian cells that can express and secrete, or that can be molecularly engineered to express and secrete, large quantities of a particular protein into the culture medium. In one embodiment, the protein produced by the cell can be endogenous or homologous to the cell. Alternatively, the protein is heterologous, i.e., foreign, to the cell.

The cells utilized in the methods of the present invention can be grown and maintained in any number of cell culture media, including those which are known in the art or are commercially available. One of ordinary skill in the art may opt to use one or more known cell culture media that is selected to maximize cell growth, cell viability, and/or protein production in a particular cultured host cell. Exemplary cell culture media include any media suitable for culturing cells that can express a protein of interest. In some embodiments, the media is chemically defined media.

Additionally, the cell culture media can optionally be supplemented to include one or more additional components, in appropriate concentrations or amounts, as necessary or desired, and as would be known and practiced by those of ordinary skill in the art. Exemplary supplements include, but are not limited to, chemical gene selection agents, hormones and other growth factors, (e.g., insulin, transferrin, epidermal growth factor, serum, somatotropin, pituitary extract, aprotinin); salts (e.g., calcium, magnesium and phosphate), and buffers (e.g., HEPES (4-[2-Hydroxethyl]-1-piperazine-ethanesulfonic acid)); nucleosides and bases (e.g., adenosine, thymidine, hypoxanthine); protein and hydrolysates; antibiotics (e.g., gentamycin); cell protective agents (e.g., a Pluronic polyol (PLURONIC® F68)) and extracellular matrix proteins (e.g., fibronectin). Supplements that support the growth and maintenance of particular cell cultures are able to be readily determined by those of ordinary skill in the art, such as is described, for example, by Barnes et al. (Cell, 22:649 (1980)); in Mammalian Cell Culture, Mather, J. P., ed., Plenum Press, NY (1984); and in U.S. Pat. No. 5,721,121.

As used herein, the term “bioreactor” refers to any apparatus, closed container or vessel (e.g., a fermentation chamber) that is used for growing cell cultures. Bioreactors allow controlling various parameters during the cell culture process including, but not limited to, the circulation loop flow, pH, the temperature, the overpressure and/or the medium perfusion rate. Bioreactors include commercially available bioreactors, classical fermenters and cell culture perfusion systems, as well as disposable bioreactors.

The bioreactor can be of any size that is useful for culturing cells at a desirable scale in accordance with a method of the invention. For example, a bioreactor employed in the methods of the present invention may be at least about 0.1, at least about 0.5, at least about 1, at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, 85, at least about 90, at least about 95, at least about 100, at least about 105, at least about 110, at least about 115, at least about 120, at least about 125, at least about 130, at least about 135, at least about 140, at least about 145, at least about 150, at least about 155, at least about 160, at least about 165, at least about 170, at least about 175, at least about 180, at least about 185, at least about 190, at least about 195, at least about 200, at least about 205, at least about 210, at least about 215, at least about 220, at least about 225, at least about 230, at least about 235, at least about 240, at least about 245, at least about 250, at least about 255, at least about 260, at least about 265, at least about 270, at least about 275, at least about 280, at least about 285, at least about 290, at least about 295, at least about 300, at least about 305, at least about 310, at least about 315, at least about 320, at least about 325, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 550, at least about 1,000, at least about 1,500, at least about 2,000, at least about 2,500, at least about 3,000, at least about 3,500, at least about 4,000, at least about 4,500, at least about 5,000, at least about 5,500, at least about 6,000, at least about 6,500, at least about 7,000, at least about 7,500, at least about 8,000, at least about 8,500, at least about 9,000, at least about 9,500, at least about 10,000, at least about 10,500, at least about 11,000, at least about 11,500, at least about 12,0000, at least about 13,000, at least about 14,000, at least about 15,000 liters or more, or any intermediate volume.

The methods of the present invention can employ one or more bioreactors. For example, in one embodiment, the perfusion cell culture and fed-batch cell culture can be performed in the same bioreactor. In another embodiment, the perfusion cell culture can be performed in separate bioreactors. In another embodiment, the cells from the perfusion cell culture can be transferred to one or more fed-batch bioreactors.

A suitable bioreactor may be composed of (i.e., constructed of) any material that is suitable for holding cell cultures under the culture conditions of the present invention and is conducive to cell growth and viability. For example, a bioreactor employed in the methods of the present invention can be made of glass, plastic or metal. However, the materials comprising the bioreactor should not interfere with expression or stability of the polypeptide product. Suitable bioreactors are known in the art and commercially available. In embodiments, the bioreactor is a N-1 seed bioreactor (N-1 bioreactor)

A perfusion bioreactor used in the methods of the present invention can be a disposable perfusion bioreactor or any other traditional perfusion bioreactors. The bioreactor may optionally be equipped with any internal or external cell retention devices, including, but not limited to, spin filters, tangential flow membrane filters, dynamic membranes, ultrasonic separators, gravity settlers, continuous centrifuge or acoustic cell retention device, microfiltration devices, ultrafiltration devices, etc.

In embodiments, the perfusion bioreactors of the invention are bioreactors capable of obtaining a high cell density and high cell viability during the perfusion process. In certain embodiments, the perfusion bioreactors are N-1 bioreactors (or N-1 seed bioreactors).

A “biomass capacitance probe” refers to a probe that can measure viable cell density, among other capabilities. A biomass capacitance probe uses capacitance to measure the total viable cells in a culture. Viable cells act as capacitors in an alternating electric field. The biomass capacitance probe can measure the charge from these cells, and report it.

The cell cultures encompassed by the methods of the present invention may be grown at any temperature appropriate for the cell type and culture conditions. In one embodiment, it is desirable to use a temperature between about 30° C. and about 38° C., to enhance protein production. In another embodiment, the temperature is at least about 25° C., least about 26° C., least about 27° C., least about 28° C., least about 29° C., least about 30° C., least about 31° C., least about 32° C., least about 33° C., least about 34° C., least about 35° C., least about 36° C., least about 37° C., least about 38° C., least about 39° C., least about 40° C., or least about 41° C. It may also be desirable to use different temperatures at different times during the culture.

### Perfusion Process

Unlike previous uses, in the instant specification, the adjustment of the constant cell-specific perfusion rate is used during the growth phase to achieve a high cell density while reducing media usage. In embodiments, the invention is directed to an optimization of a perfusion culture process. In particular embodiments, the perfusion process takes places in an N-1 bioreactor. In an N-1 bioreactor, there is an intense period of growth in the perfusion bioreactor prior to the transfer of the cells into the production bioreactor, and cells grown in an N-1 bioreactor are capable of being grown to a high cell density.

In certain embodiments, the perfusion bioreactor is about a 1 L, 5 L, 10 L, 50 L, 100 L, 200 L, 300 L, or 500 L bioreactor. In particular embodiments, the perfusion bioreactor is a 200 L bioreactor. In certain embodiments, the perfusion bioreactor is a 5 L bioreactor.

In certain embodiments, the present invention uses an alternating tangential flow (or ATF) with hollow fiber filter to exchange media while retaining cells in bioreactor. In particular embodiments, the ATF System is attached to the N-1 bioreactor. In the ATF system, a diaphragm pump coupled with an air and vacuum system draws bioreactor contents (cells and media) into the hollow fiber filter, and then pushes the culture back into the bioreactor while simultaneously passing the permeate (cell-free media) through the hollow fiber filter. The mechanism of this alternating tangential flow through the hollow fibers is driven by a diaphragm pump and an external controller. A separate pump is attached to the ATF device to continuously draw the permeate and this is equal to the perfusion rate. Another pump is attached to the bioreactor inlet to feed fresh media to maintain the bioreactor at a target volume.

In certain embodiments, the perfusion process is optimized by measurement of the viable cell density in the culture. In particular embodiments, the measurement of the viable cell density is used to adjust the perfusion rate to meet a target cell-specific perfusion rate or a target perfusion or permeate flow rate.

In embodiments, the viable cell density is measured using a biomass capacitance probe, also called an online capacitance probe. By utilizing a biomass capacitance probe that has the reliability and accuracy of predicting online cell densities in the bioreactor, the perfusion rate can be dynamically adjusted to meet a target perfusion rate constantly, thereby reducing media usage while providing constant levels of nutrients. In certain embodiments, the biomass capacitance probe is the Hamilton Incyte biomass capacitance probe. In particular embodiments, the biomass capacitance probe is the Incyte LC. In other embodiments, the biomass capacitance probe is the Incyte HC. The Incyte measures online permittivity, and provides information only on viable cells in the reactor. The information provided by the Incyte can be used to correlate to viable cell density.

In certain embodiments, the perfusion rate is dynamically adjusted based on the measurement of the viable cell density. In certain embodiments, the target perfusion rate is calculated using the viable cell density and the constant cell-specific perfusion rate, as seen in Equation 1. In embodiments, the constant cell-specific perfusion rate is between about 0.01 to about 0.2 nL/cell/day, about 0.01 to about 0.15 nL/cell/day, about 0.02 to about 0.10 nL/cell/day, about 0.02 to about 0.05 nl/cell/day, or about 0.03 to about 0.05 nl/cell/day. In particular embodiments, the constant cell-specific perfusion rate is about 0.01 nL/cell/day, about 0.02 nL/cell/day, about 0.03 nL/cell/day, about 0.04 nL/cell/day, about 0.05 nl/cell/day, about 0.06 nL/cell/day, about 0.07 nL/cell/day, about 0.08 nL/cell/day, about 0.09 nL/cell/day, about 0.10 nL/cell/day, about 0.11 nL/cell/day, about 0.12 nL/cell/day, about 0.13 nL/cell/day, about 0.14 nL/cell/day, or about 0.15 nL/cell/day. In certain embodiments, the constant cell-specific perfusion rate is about 0.04 nL/cell/day. In other embodiments, the constant cell-specific perfusion rate is about 0.03 nL/cell/day. In yet other embodiments, the constant cell-specific perfusion rate is about 0.02 nL/cell/day. In specific embodiments, the target perfusion rate is calculated using a bioreactor controller.

In certain embodiments, as a result of the dynamic measurement of the VCD and the use of a target perfusion rate, the perfusion reactor uses less than 5 vessel volumes of perfusion media per day. In particular embodiments, the perfusion reactor uses less than about 5, less than about 4.5, less than about 4, less than about 3.5, less than about 3.0, less than about 2.5, less than about 2, less than about 1.5 or less than 1 vessel volumes of perfusion media per day. In certain embodiments, as a result of the dynamic measurement of the VCD and the use of a target perfusion rate, the perfusion reactor uses only about 25%, about 30%, about 35%, about 40%, about 45%, about 50%, about 55%, about 60%, about 65%, about 70%, about 75%, about 80%, about 85%, about 90%, or about 95% of the total amount of perfusion media used by a process that does not dynamically measure the VCD.

In embodiments, the biomass capacitance probe is used to determine the critical time point when the cell density in the bioreactor reaches a target cell density and is transferred to a production bioreactor. In some embodiments, the target cell density is a high cell density. In certain embodiments, the target cell density is at least about 40×10⁶ cells/ml, about 50×10⁶ cells/ml, about 60×10⁶ cells/ml, about 70×10⁶ cells/ml, about 80×10⁶ cells/ml, about 90 x 10⁶ cells/ml, or about 100×10⁶ cells/ml. In particular embodiments, the target cell density is at least about 60×10⁶ cells/ml. In certain embodiments, the target cell density is greater than about 60×10⁶ cells/ml.

In embodiments, the pH in the perfusion bioreactor is maintained at about 6.0 to about 8.0, about 6.5 to about 7.5, about 6.6 to about 7.5, about 6.7 to about 7.5, about 6.8 to about 7.5, or about 6.8 to about 7.4. In certain embodiments, the pH in the perfusion bioreactor is maintained at about 6.0, about 6.1, about 6.2, about 6.3, about 6.4, about 6.5, about 6.6, about 6.7, about 6.8, about 6.9, about 7.0, about 7.1, about 7.2, about 7.2, about 7.4, about 7.5, about 7.6, about 7.7, about 7.8, about 7.9 or about 8.0.

In particular embodiments, the cells are transferred from the perfusion bioreactor to a production bioreactor once the target cell density is released. In embodiments, the cells are transferred to a production bioreactor that is about a 200 L, 500 L, 1000 L, 1500 L, 2000 L, 3000 L or larger production bioreactor. In certain embodiments, the production bioreactor is inoculated with the perfusion culture at a higher cell density. In embodiments, the production bioreactor is inoculated at a cell density of about 1×10⁶ cells/ml, about 2×10⁶ cells/ml, about 3×10⁶ cells/ml, about 4×10⁶ cells/ml, about 5×10⁶ cells/ml, about 6×10⁶ cells/ml, about 7×10⁶ cells/ml, about 8×10⁶ cells/ml, about 9×10⁶ cells/ml, about 10×10⁶ cells/ml, about 11×10⁶ cells/ml, about 12×10⁶ cells/ml, about 13×10⁶ cells/ml, about 14 x 10⁶ cells/ml, about 15×10⁶ cells/ml, about 20×10⁶ cells/ml, about 30×10⁶ cells/ml, about 40×10⁶ cells/ml, about 50×10⁶ cells/ml, or a greater cell density. In particular embodiments, the production bioreactor is inoculated at a cell density of about 10×10⁶ cells/ml. In particular embodiments, the production bioreactor is inoculated at a cell density of about 5×10⁶ cells/ml. In other embodiments, the production bioreactor is inoculated at a cell density of about 6×10⁶ cells/ml.

In embodiments, the temperature in the bioreactor is maintained at about 34 ° C., about 34.5° C., about 35° C., about 35.5° C., about 36 ° C., about 36.5 ° C., about 37 ° C., about 37.5 ° C., about 38° C., about 38.5° C., or about 39° C. [72] In some embodiments, the total perfusate is measured. In embodiments, the daily perfusion rate (bioreactor volume) is measured. In certain embodiments, the capacitance is measured.

### Polypeptides

Any polypeptide that is expressible in a host cell may be produced in accordance with the present invention. The polypeptide may be expressed from a gene that is endogenous to the host cell, or from a gene that is introduced into the host cell through genetic engineering. The polypeptide may be one that occurs in nature, or may alternatively have a sequence that was engineered or selected by the hand of man. An engineered polypeptide may be assembled from other polypeptide segments that individually occur in nature, or may include one or more segments that are not naturally occurring.

Polypeptides that may desirably be expressed in accordance with the present invention will often be selected on the basis of an interesting biological or chemical activity. For example, the present invention may be employed to express any pharmaceutically or commercially relevant enzyme, receptor, antibody, hormone, regulatory factor, antigen, binding agent, etc.

### Antibodies

Given the large number of antibodies currently in use or under investigation as pharmaceutical or other commercial agents, production of antibodies is of interest in accordance with the present invention. Antibodies are proteins that have the ability to specifically bind a particular antigen. Any antibody that can be expressed in a host cell may be used in accordance with the present invention. In an embodiment, the antibody to be expressed is a monoclonal antibody. In certain embodiments, the antibody is a polyclonal antibody.

In another embodiment, the antibody is a chimeric antibody. A chimeric antibody contains amino acid fragments that are derived from more than one organism. Chimeric antibody molecules can include, for example, an antigen binding domain from an antibody of a mouse, rat, or other species, with human constant regions. A variety of approaches for making chimeric antibodies have been described. See e.g., Morrison et al., Proc. Natl. Acad. Sci. U.S.A. 81, 6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B.

In another embodiment, the antibody is human antibody derived, e.g., through the use of ribosome-display or phage-display libraries (see, e.g., Winter et al., U.S. Pat. No. 6,291,159 and Kawasaki, U.S. Pat. No. 5,658,754) or the use of xenographic species in which the native antibody genes are inactivated and functionally replaced with human antibody genes, while leaving intact the other components of the native immune system (see, e.g., Kucherlapati et al., U.S. Pat. No. 6,657,103).

Five human immunoglobulin classes are defined on the basis of their heavy chain composition, and are named IgG, IgM, IgA, IgE, and IgD. The IgG-class and IgA-class antibodies are further divided into subclasses, namely, IgG1, IgG2, IgG3, and IgG4, and IgAl and IgA2. The heavy chains in IgG, IgA, and IgD antibodies have three constant region domains, that are designated CH1, CH2, and CH3, and the heavy chains in IgM and IgE antibodies have four constant region domains, CH1, CH2, CH3, and CH4.

In another embodiment, the antibody is a humanized antibody. A humanized antibody is a chimeric antibody wherein the large majority of the amino acid residues are derived from human antibodies, thus minimizing any potential immune reaction when delivered to a human subject. In humanized antibodies, amino acid residues in the complementarity determining regions are replaced, at least in part, with residues from a non-human species that confer a desired antigen specificity or affinity. Such altered immunoglobulin molecules can be made by any of several techniques known in the art, (e.g., Teng et al., Proc. Natl. Acad. Sci. USA., 80, 7308-7312 (1983); Kozbor et al., Immunology Today, 4, 7279 (1983); Olsson et al., Meth. Enzymol., 92, 3-16 (1982)), and are made according to the teachings of PCT Publication W092/06193 or EP 0239400, all of which are incorporated herein by reference). Humanized antibodies can also be commercially produced. For further reference, see Jones et al., Nature 321:522-525 (1986); Riechmann et al., Nature 332:323-329 (1988); and Presta, Curr. Op. Struct. Biol. 2:593-596 (1992), all of which are incorporated herein by reference.

In still another embodiment, the monoclonal, polyclonal, chimeric, or humanized antibodies described above may contain amino acid residues that do not naturally occur in any antibody in any species in nature. These foreign residues can be utilized, for example, to confer novel or modified specificity, affinity or effector function on the monoclonal, chimeric or humanized antibody. In another embodiment, the antibodies described above may be conjugated to drugs for systemic pharmacotherapy, such as toxins, low-molecular-weight cytotoxic drugs, biological response modifiers, and radionuclides (see e.g., Kunz et al., Calicheamicin derivative-carrier conjugates, US20040082764 A1).

### Cells

Any mammalian cell or cell type susceptible to cell culture, and to expression of polypeptides, may be utilized in accordance with the present invention. Non-limiting examples of mammalian cells that may be used in accordance with the present invention include BALB/c mouse myeloma line (NSW, ECACC No: 85110503); human retinoblasts (PER.C6 (CruCell, Leiden, The Netherlands)); monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells±DHFR (CHO, Urlaub and Chasin, Proc. Natl. Acad. Sci. USA, 77:4216 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCLS 1); TM cells (Mather et al., Annals N.Y. Acad. Sci., 383:44-68 (1982)); MRC 5 cells; FS4 cells; and a human hepatoma line (Hep G2). In a particularly embodiment, the present invention is used in the culturing of and expression of polypeptides and proteins from CHO cell lines.

Additionally, any number of commercially and non-commercially available hybridoma cell lines that express polypeptides or proteins may be utilized in accordance with the present invention. One skilled in the art will appreciate that hybridoma cell lines might have different nutrition requirements and/or might require different culture conditions for optimal growth and polypeptide or protein expression, and will be able to modify conditions as needed.

As noted above, in many instances the cells will be selected or engineered to produce high levels of protein or polypeptide. Often, cells are genetically engineered to produce high levels of protein, for example by introduction of a gene encoding the protein or polypeptide of interest and/or by introduction of control elements that regulate expression of the gene (whether endogenous or introduced) encoding the polypeptide of interest.

Certain polypeptides may have detrimental effects on cell growth, cell viability or some other characteristic of the cells that ultimately limits production of the polypeptide or protein of interest in some way. Even amongst a population of cells of one particular type engineered to express a specific polypeptide, variability within the cellular population exists such that certain individual cells will grow better and/or produce more polypeptide of interest. In certain embodiments of the present invention, the cell line is empirically selected by the practitioner for robust growth under the particular conditions chosen for culturing the cells. In particular embodiments, individual cells engineered to express a particular polypeptide are chosen for large-scale production based on cell growth, final cell density, percent cell viability, titer of the expressed polypeptide or any combination of these or any other conditions deemed important by the practitioner.

### Media and Culture Conditions

In some embodiments, a mammalian host cell is cultured under conditions that promote the production of the polypeptide of interest, any polypeptide disclosed herein. Basal cell culture medium formulations are well known in the art. To these basal culture medium formulations the skilled artisan will add components such as amino acids, salts, sugars, vitamins, hormones, growth factors, buffers, antibiotics, lipids, trace elements and the like, depending on the requirements of the host cells to be cultured. The culture medium may or may not contain serum and/or protein. Various tissue culture media, including serum-free and/or defined culture media, are commercially available for cell culture. Tissue culture media is defined, for purposes of the invention, as a media suitable for growth of animal cells, and, in some embodiments, mammalian cells, in in vitro cell culture. Typically, tissue culture media contains a buffer, salts, energy source, amino acids, vitamins and trace essential elements. Any media capable of supporting growth of the appropriate eukaryotic cell in culture can be used; the invention is broadly applicable to eukaryotic cells in culture, particularly mammalian cells, and the choice of media is not crucial to the invention. Tissue culture media suitable for use in the invention are commercially available from, e.g., ATCC (Manassas, Va.). For example, any one or combination of the following media can be used: RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL.TM. 300 Series (available from JRH Biosciences, Lenexa, Kans., USA), among others, which can be obtained from the American Type Culture Collection or JRH Biosciences, as well as other vendors. When defined medium that is serum-free and/or peptone-free is used, the medium is usually highly enriched for amino acids and trace elements. See, for example, U.S. Pat. No. 5,122,469 to Mather et al. and U.S. Pat. No. 5,633,162 to Keen et al.

In certain embodiments, cells can be grown in serum-free, protein-free, growth factor-free, and/or peptone-free media. The term “serum-free” as applied to media includes any mammalian cell culture medium that does not contain serum, such as fetal bovine serum. The term “insulin-free” as applied to media includes any medium to which no exogenous insulin has been added. By exogenous is meant, in this context, other than that produced by the culturing of the cells themselves. The term “IGF-1-free” as applied to media includes any medium to which no exogenous Insulin-like growth factor-1 (IGF-1) or analog (such as, for example, LongR3, [Ala31], or [Leu24][Ala31] IGF-1 analogs available from GroPep Ltd. of Thebarton, South Australia) has been added. The term “growth-factor free” as applied to media includes any medium to which no exogenous growth factor (e.g., insulin, IGF-1) has been added. The term “protein-free” as applied to media includes medium free from exogenously added protein, such as, for example, transferrin and the protein growth factors IGF-1 and insulin. Protein-free media may or may not have peptones. The term “peptone-free” as applied to media includes any medium to which no exogenous protein hydrolysates have been added such as, for example, animal and/or plant protein hydrolysates. Eliminating peptone from media has the advantages of reducing lot to lot variability and enhancing processing such as filtration. Chemically defined media are media in which every component is defined and obtained from a pure source, in certain embodiments, a non-animal source. In certain embodiments, the media is chemically defined and fully serum and protein free.

In some embodiments, one of the many individualized media formulations that have been developed to maximize cell growth, cell viability, and/or recombinant polypeptide production in a particular cultured host cell is utilized. The methods described herein may be used in combination with commercially available cell culture media or with a cell culture medium that has been individually formulated for use with a particular cell line. For example, an enriched medium that could support increased polypeptide production may comprise a mixture of two or more commercial media, such as, for instance, DMEM and Ham's F1 2 media combined in ratios such as, for example, 1:1, 1:2, 1:3, 1:4, 1:5, 1:6, 1:7, 1:8, or even up to 1:15 or higher. Alternatively or in addition, a medium can be enriched by the addition of nutrients, such as amino acids or peptone, and/or a medium (or most of its components with the exceptions noted below) can be used at greater than its usual, recommended concentration, for example at 2×, 3×, 4×, 5×, 6×, 7×, 8×, or even higher concentrations. As used herein, “1×” means the standard concentration, “2×” means twice the standard concentration, etc. In any of these embodiments, medium components that can substantially affect osmolarity, such as salts, cannot be increased in concentration so that the osmolarity of the medium falls outside of an acceptable range. Thus, a medium may, for example, be 8× with respect to all components except salts, which can be present at only 1×. An enriched medium may be serum free and/or protein free. Further, a medium may be supplemented periodically during the time a culture is maintained to replenish medium components that can become depleted such as, for example, vitamins, amino acids, and metabolic precursors. As is known in the art, different media and temperatures may have somewhat different effects on different cell lines, and the same medium and temperature may not be suitable for all cell lines.

Suitable culture conditions for mammalian cells are known in the art. See e.g. Animal cell culture: A Practical Approach, D. Rickwood, ed., Oxford university press, New York (1992). Mammalian cells may be cultured in suspension or while attached to a solid substrate. Furthermore, mammalian cells may be cultured, for example, in fluidized bed bioreactors, hollow fiber bioreactors, roller bottles, shake flasks, or stirred tank bioreactors, with or without microcarriers, and operated in a batch, fed batch, continuous, semi-continuous, or perfusion mode.

### Monitoring Culture Conditions

In certain embodiments of the present invention, the practitioner may find it beneficial or necessary to periodically monitor particular conditions of the growing cell culture. Monitoring cell culture conditions allows the practitioner to determine whether the cell culture is producing recombinant polypeptide or protein at suboptimal levels or whether the culture is about to enter into a suboptimal production phase. In order to monitor certain cell culture conditions, it will be necessary to remove small aliquots of the culture for analysis. One of ordinary skill in the art will understand that such removal may potentially introduce contamination into the cell culture, and will take appropriate care to minimize the risk of such contamination.

As non-limiting example, it may be beneficial or necessary to monitor temperature, pH, cell density, cell viability, integrated viable cell density, lactate levels, ammonium levels, osmolarity, or titer of the expressed polypeptide or protein. Numerous techniques are well known in the art that will allow one of ordinary skill in the art to measure these conditions. For example, cell density may be measured using a hemacytometer, a Coulter counter, or Cell density examination (CEDEX). Viable cell density may be determined by staining a culture sample with Trypan blue.. Since only dead cells take up the Trypan blue, viable cell density can be determined by counting the total number of cells, dividing the number of cells that take up the dye by the total number of cells, and taking the reciprocal. Cell viability can also be measured using a biomass capacitance probe. HPLC, UPLC, NOVA Flex or Cedex Bio can be used to determine the levels of lactate, ammonium or the expressed polypeptide or protein. Alternatively, the level of the expressed polypeptide or protein can be determined by standard molecular biology techniques such as coomassie staining of SDS-PAGE gels, Western blotting, Bradford assays, Lowry assays, Biuret assays, and UV absorbance. It may also be beneficial or necessary to monitor the post-translational modifications of the expressed polypeptide or protein, including phosphorylation and glycosylation.

### EXAMPLES

### Example 1

The following example describes the use of a biomass capacitance probe applied in an N-1 bioreactor for:
1. Measuring online VCD (viable cell densities). VCD and a constant cell-specific perfusion rate (between 0.02 to 0.10 nL/cell/day) were utilized to determine the amount of media to pump out from the bioreactor (this is termed the target perfusion or permeate flow rate). This calculation is done by a bioreactor controller. A separate flow sensor measured the flow rate and either increase or decrease the perfusion pump flow rate to achieve the target flow rate.
2. Estimating with a higher degree of accuracy the critical time point to inoculate production vessels by measuring online VCD with more datapoints. At this critical time point, sufficient cells were ready to transferred out from a N-1 perfusion bioreactor to inoculate into a production bioreactor vessel at a higher cell densities (>5 E6 cells/ml)

The following experiment describes this method that has been applied to a N-1 seed bioreactor at the 5-L and 200-L bioreactor scale.

### Cell Line and Cell Culture Medium

A BMS-proprietary cell culture medium (B6) and five proprietary recombinant CHO cell lines expressing different IgG antibodies were used in these experiments. The cell culture medium is chemically defined and fully serum and protein free. Recombinant IgG producing CHO cells were maintained in suspension culture in 250-mL, 1-L and 3L shake flasks. A CO₂ shaker incubator (Kuhner) was used for incubation at 36.5° C., 150 rpm at a CO₂ concentration of 5%

### Bench-Scale Bioreactors and Cultivation Conditions

Bench scale reactor cultivations for both N-1 seed and Fed-batch production were carried out in 5-L stirred tank reactors (Sartorius). All of the 5-L reactors were equipped with two 45° pitched tri-blade impellers, pH, DO and biomass capacitance probe. In the N-1 seed, cultivations were inoculated from 3-L shake flask cultures in the range of 1-2×10⁶ cells/ml and at a temperature of 36.5° C. The working volume of the 5-L bioreactor was at 3 L. PH was controlled in the range of 6.8 to 7.4 by 1M sodium carbonate addition and CO₂ gas sparging. Impeller agitation was set at 240 rpm, aeration was provided by pure oxygen sparging through 0.5 mm drilled hole spargers. Dissolved oxygen was maintained at a level of 40% via cascade oxygen sparging. When oxygen sparging reaches a maximum sparge rate of 0.5 lpm, additional oxygen sparge was directed through 10 p.m spargers. Antifoam (EX-CELL antifoam, Sigma-Aldrich) was added to the bioreactor to control foam levels. Cell density was measured by daily offline measurements (Vi-cell, Beckman Coulter) and/ or via online capacitance probes (Hamilton). Daily offline samples was also monitored for pH, dissolved oxygen and pCO2 via phox instruments (Nova Biomedical) and glucose, lactate profiles were measured with Cedex Bio HT (Roche). [95] In the fed-batch production bioreactor, cells were inoculated from the perfusion N-1 seed culture from the 5-L production vessel at a cell density of either 6×10⁶ or 10×10⁶ cells/ml. Bolus feeds of chemically defined media were added to the production vessel starting from day 0-2. Amounts of feed varies between 3 to 6% of initial working volume. The agitation and sparger control strategies are similar to the N-1 seed bioreactor described above. Cell densities, metabolite profiles were measured using the same methods described above. Additional 10 ml supernatant samples were also obtained from the fed-batch production bioreactor. After centrifugation for 5 min at 1000 g , cell-free samples were frozen at −80 C before measurement of IgG titer by UPLC methods. 200 L bioreactors and 500 L production bioreactors

Bench scale reactor cultivations for N-1 seed and Fed-batch production were carried out in 200-L and 500-L single use bioreactor (SUB) bags (GE Xcellerex) respectively. The SUBs were equipped with a bottom mounted, magnetic driven impeller, and pH, DO, biomass capacitance probes. In the N-1 seed, cultivations were inoculated from one or more 50-L Wave bags in the range of 1-2×10⁶ cells/ml and at a temperature of 36.5° C. The working volume of the 200-L SUB was between 130-200 L. PH was controlled in the range of 6.8 to 7.4 by 1M sodium carbonate addition and CO₂ gas sparging. Impeller agitation was set between 80-110 rpm, aeration was provided by pure oxygen sparging through 1 mm disc spargers. Dissolved oxygen was maintained at a level of 40% via cascade oxygen sparging.. Antifoam (EX-CELL antifoam, Sigma-Aldrich) was added to the bioreactor to control foam levels. Cell density was measured by daily offline measurements (Vi-cell, Beckman Coulter) and/ or via online capacitance probes (Hamilton). Daily offline samples was also monitored for pH, dissolved oxygen and pCO2 via phox instruments (Nova Biomedical) and glucose, lactate profiles were measured with Cedex Bio HT (Roche).

In the fed-batch production bioreactor, cells were inoculated from the perfusion N-1 seed culture from the 200-L production vessel at a cell density of either 6×10⁶ or 10×10⁶ cells/ml. Bolus feeds of chemically defined media were added to the production vessel starting from day 0-2. Amounts of feed varies between 3 to 6% of initial working volume. Cell densities, metabolite profiles were measured using the same methods described above. 10 ml supernatant samples were also obtained from the fed-batch production bioreactor. After centrifugation for 5 min at 1000 g , cell-free samples were frozen at −80 C before measurement of IgG titer by UPLC methods.

### Control of Perfusion Rate in the ATF2 in Bench Scale Bioreactors

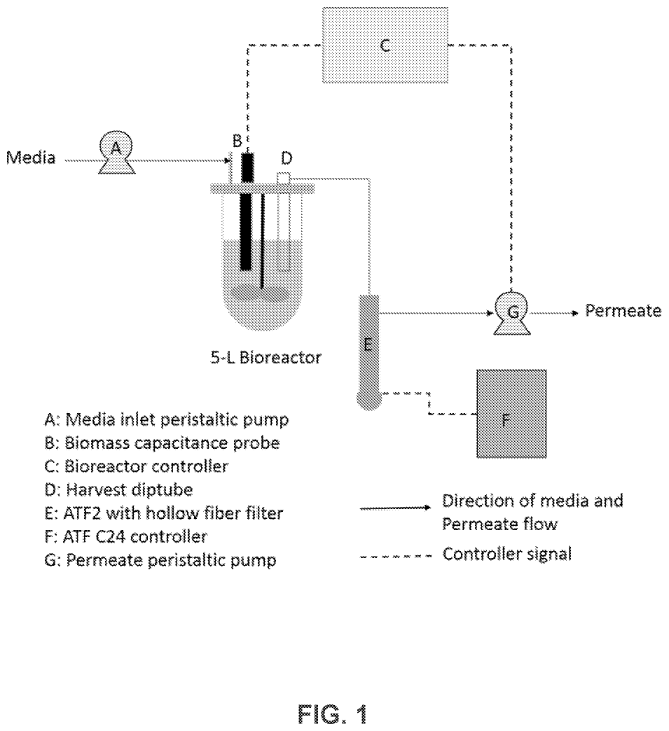

Cells were inoculated into 5-L bioreactors at 1-1.5×10⁶ cells/ml and started in batch mode and later switched to perfusion operation when cell densities reach a range of 3-8×10⁶ cells/ml. The ATF 6 system was used to operate perfusion, and the device includes a polyethersulfone or polysulfone 0.2 um hollow fiber filter as the cell retention device. An ATF controller was connected to the bottom hemisphere of the ATF system to control the ATF flow rate via a diaphragm pump (Repligen). Other parameters of the ATF6 hollow fiber filter is described in Table 1. Two peristaltic pumps were attached to the bioreactor: one controls the media inlet into the bioreactor and the second pump controls the permeate from the ATF, which is equivalent to perfusion rate (See FIG. 1).

The permeate pump rate was controlled by a Finesse controller output, to maintain a target flow rate. This target flow rate was determined by equation 1, with CSPR, working volume and online VCD profiles measured by biomass capacitance probe. In order to maintain a constant bioreactor working volume the vessel weight was monitored on a scale, and the media inlet peristaltic pump will feed the bioreactor with fresh media to maintain a constant weight. This perfusion operation continued until a target viable cell density of >40×10⁶ cells/ml was reached and then transferred to a production vessel at a high density.

As a control, the permeate pump rate was also set manually to a specified perfusion rate that is based on the following settings: when cell densities reached between 3-8 million, perfusion rate was set at 1 volumes of the working volume of the reactor, the perfusion rate was increased daily as needed to ensure the culture maintained a perfusion rate of 0.04 nL/cell/day based on the offline VCD values.

$$Permeate Pump SP ( mL / min ) = \frac{\begin{matrix}Online VCD ( \frac{{10}^{6} cells}{ml} ) \times CSPR ( \frac{mL}{{10}^{6} cell * day} ) \\ Working Volume ( mL )\end{matrix} \times }{1440 ( min / day )} \qquad Equation 1$$

Where Online VCD=Cell Factor×Biocapacitance reading, Cell factor is derived from correlation of offline VCD to biocapacitance readings (pF)

### Control of Perfusion Rate in the ATF6 and 200 L Bioreactors

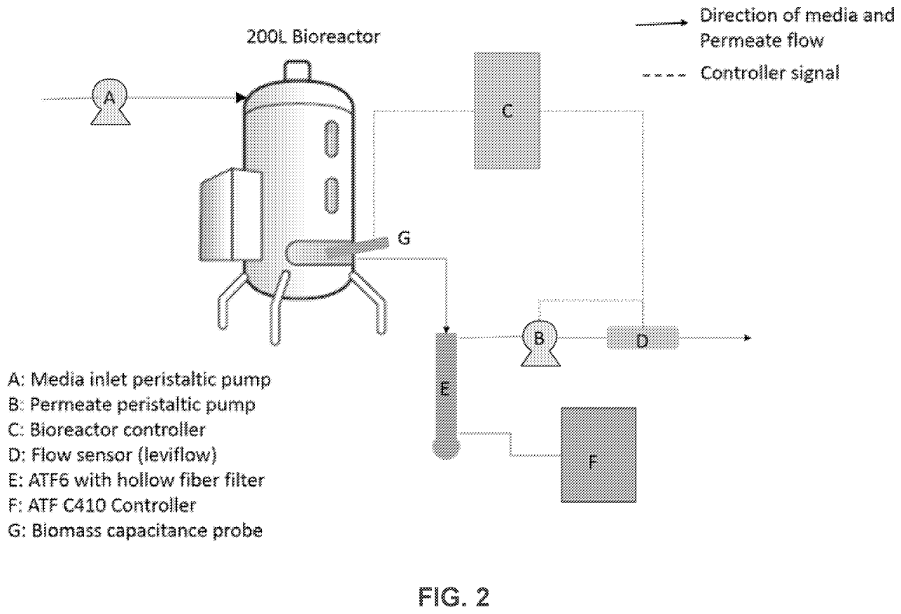

Cells were inoculated into 200-L bioreactors and operated in batch mode until cell densities reach 3-8×10⁶ cells/ml, upon which perfusion operation was initiated. The ATF 6 system included a polyethersulfone or polysulfone 0.2 um hollow fiber filter as the cell retention device (either stainless steel or single use). An ATF controller was connected to the bottom hemisphere of the ATF system to control the ATF flow rate via a diaphragm pump (Repligen). Other parameters of the ATF2 hollow fiber filter was described in Table 1.Two peristaltic pumps were attached to the bioreactor: one controls the media inlet into the bioreactor and other controls the permeate from the ATF (See FIG. 2). The permeate line was also connected to a flow sensor (leviflow) to measure and control flow rate of the permeate pump. The media-in line was connected to a separate peristaltic pump and fed into a top port of the SUB.

The permeate flow rate was equivalent to the perfusion rate and was controlled by the Xcellerex controller based on a flow rate setpoint verified by a flow sensor (Leviflow). The target flow rate was based on equation 1. The Leviflow sensor measured the permeate flow rate downstream of the pump and drives the permeate peristaltic pump to the targeted flow rate determined by equation 1. In order to maintain a constant bioreactor working volume the vessel weight was monitored on a load-cell media is fed through the peristaltic pump in the media inlet line to maintain a constant weight. The perfusion operation continued until a target viable cell density of >40×10⁶ cells/ml was reached and then transferred to a production vessel at a high density.

TABLE 1
Description of hollow fiber filters and alternating
tangential flow rates in ATF-6 used in 5-L bioreactor
and ATF-6 used in the 200-L bioreactor

| | ATF 2 | ATF 6 |
|---|---|---|
| Filter size (μm) | 0.2 | 0.2 |
| Effective filter surface area | 0.085 | 2.1 |
| (m²) | | |
| Number of hollow fiber | 50 | 1250 |
| tubings | | |
| ATF rate (LPM) | 0.8 | 17.3 |
| Volume displaced through | 0.09-0.1 | 1.3-1.5 |
| hollow fiber tubings per | | |
| cycle (L) | | |

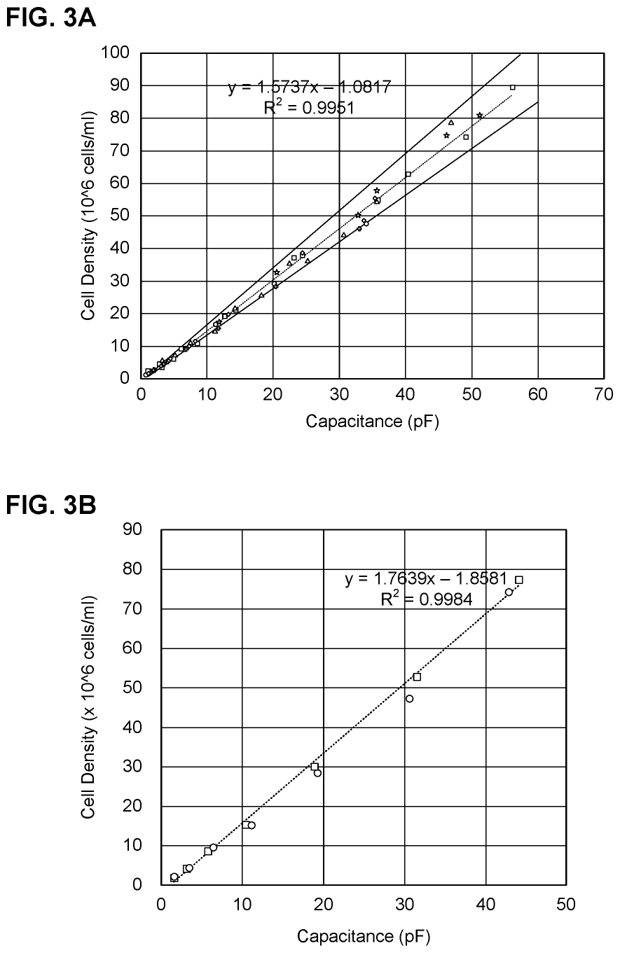

FIG. 3 shows the correlation of viable cell densities to biomass capacitance readings. The correlation is linear with an R square value above 0.95. The slope in the correlation VCD to capacitance is used as the cell factor that is described in equation 1. Hence this capacitance signal is used to determine the perfusion rate or permeate flow rate for the ATF. As shown in FIG. 3, the two cell lines (Cells lines A and B) produced different IgG entities, yield different cell factor equations.

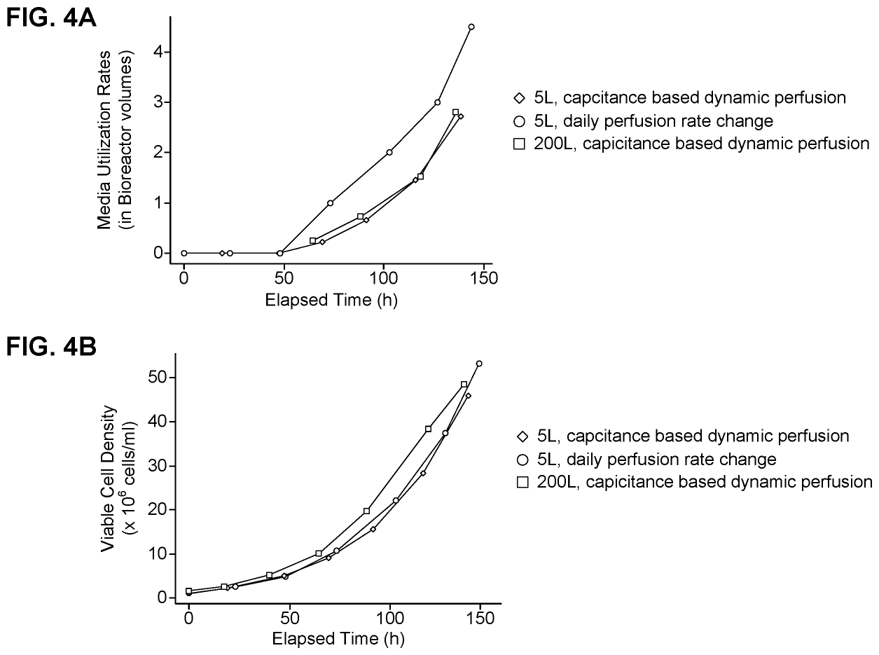

FIG. 4 shows media utilization rates for the N-1 bioreactor operated at the two different modes of perfusion for cell line A. The dynamic control of perfusion rate based on bio-capacitance reading is highlighted as diamondafor bench—scale 5 L and pilot plant scale 200 L. A perfusion rate based on daily perfusion rate changes is represented by circles. The data shows overall ˜30% less media was used in this method but yielded a process with similar cell density growth rates. Thus, dynamic control of perfusion rate was demonstrated both in the 5-L as well as the 200-L scale.

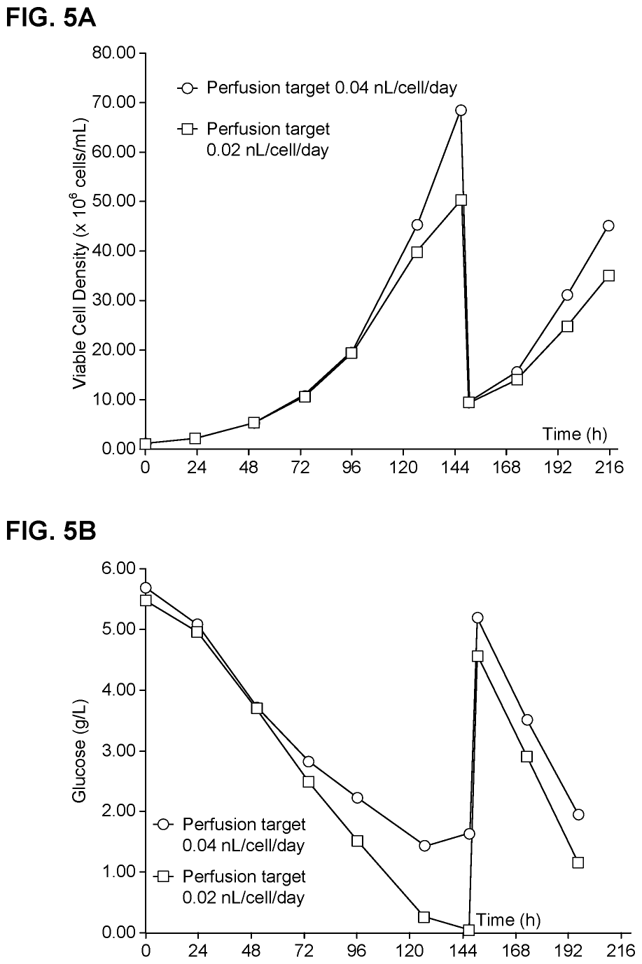

The method of dynamically controlling perfusion rate based on online VCD readings was also used to test different cell-specific perfusion rates and the impact to the N-1 seed growth. In FIG. 5, two different cell-specific perfusion rates (CSPR) were tested for cell line C. At the lower CSPR of 0.02 nL/cell-day, cell growth was reduced after 120 hours, and a lower final VCD was achieved on 144 hours. The CSPR of 0.02 nL/cell-day also results in lower glucose concentration and almost depletion on 144 hours. Therefore, this method can be used to determine an optimal CSPR perfusion rates for the N-1 perfusion process.

The perfusion N-1 seed described above was subsequently inoculated into high seed density fed-batch production bioreactors at cell densities of 6 and 10×10⁶ cells/ml respectively. Irrespective of CSPR rates in the N-1 seed, all bioreactors reached similar levels of final titer. Bioreactors that are inoculated from a seed at higher CSPR rates of 0.04 nL/cell/day achieved a higher peak VCD on Day 4.

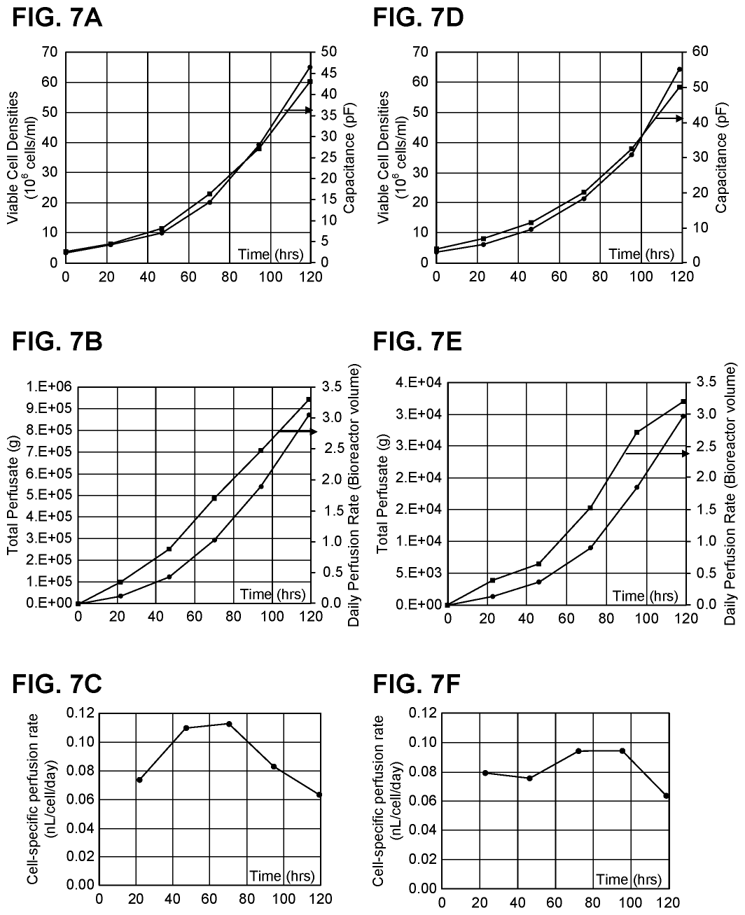

The selection of a target cell-specific perfusion rate (CSPR) at the N-1 seed stage is dependent on the cell line and the growth medium in which it is cultivated. Using the standard seed expansion growth medium developed in-house by BMS, cell line D requires a CSPR of 0.08-0.10 nL/cell-day to achieve a doubling time of 27-28 hours, and reached a cell density of 64 E6 cells/ml on Day 5 (FIG. 7). This was demonstrated in both the 5-L and 200-L bioreactor. For this cell line, approximately 9 bioreactor volumes of growth medium was used in this N-1 seed stage.

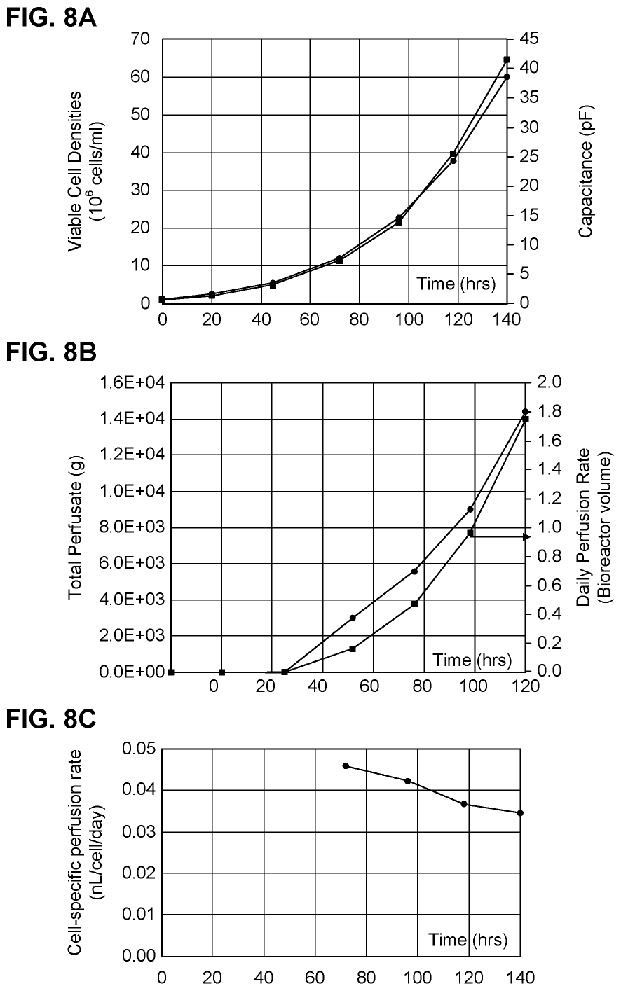

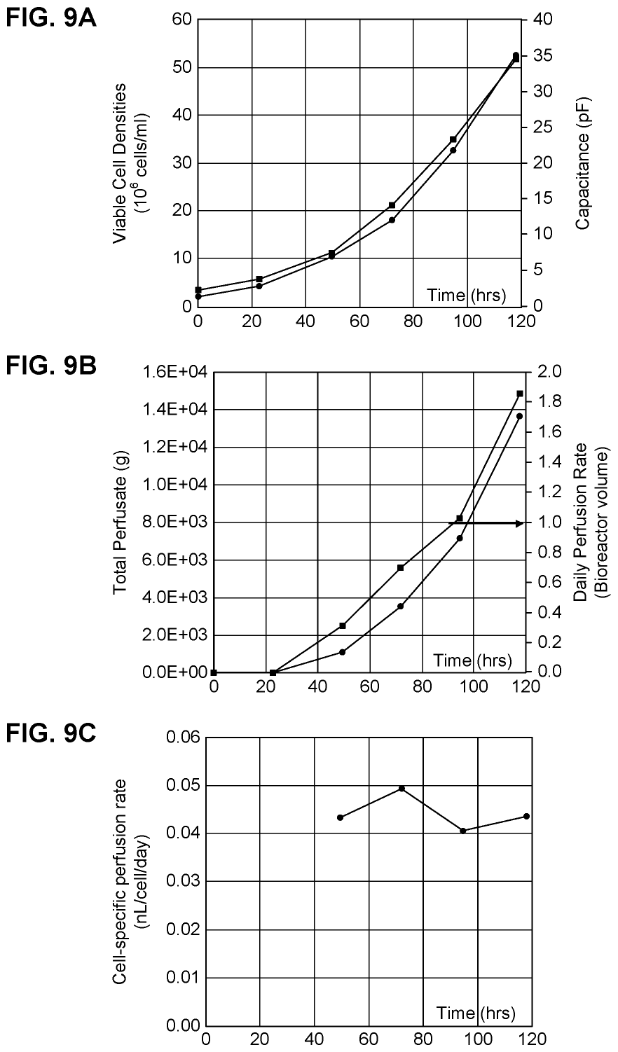

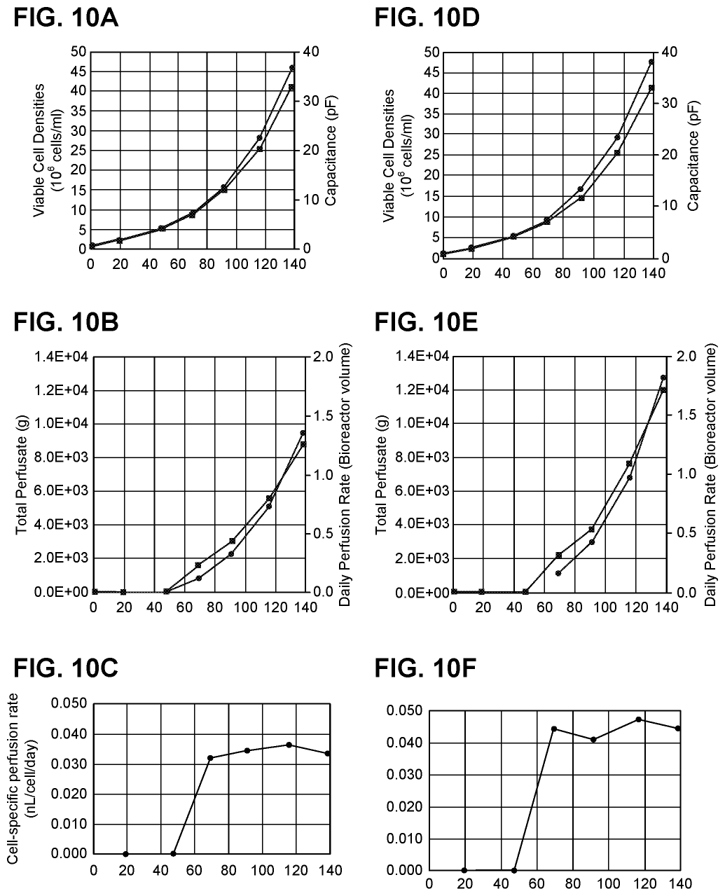

In a different study using the same growth medium, cell line C requires only a CSPR of 0.04-0.05 nL/cell-day to achieve a doubling time of 24.0 hours , and reached a final cell density of 64.6 E6 cells/ml on Day 6 (FIG. 8). For cell line C, approximately 4 bioreactor volumes of growth medium was used in N-1 seed stage. At the same CSPR of 0.04-0.05 nL/cell-day, cell line E achieved a doubling time of 24.6 hours, and reached a final cell density of 52.7 E6 cells/ml on Day 6 (FIG. 9). In cell line A, two different CSPR rates of 0.03 and 0.04 nL/cell-day was evaluated. The different CSPR did not impact cell growth rate, however controlling perfusion at the lower CSPR of 0.03 nL/cell-day, reduced the total perfusate volume by 20%. (FIG. 10)

The lowest CSPR required to achieve a final VCD of >50 E6 cells/ml is preferred to lower the cost of goods, and minimize the volumes of media utilized in the N-1 seed stage. However, a CSPR that is set too low could also impact growth rate and results in nutrient depletion as shown in FIG. 5 (Cell line C). CSPR is set empirically and can vary depending on the type of growth medium.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

1. A method of controlling the perfusion rate in a perfusion bioreactor comprising:
a) measuring the viable cell density of cells growing in cuture using a biomass capacitance probe; and
b) dynamically adjusting the target perfusion rate in view of the viable cell density, wherein the cells are grown to a high cell density during perfusion.

2. A method of minimizing cell culture medium perfusion volume comprising:
a) measuring the viable cell density of cells growing in cuture using a biomass capacitance probe; and
b) dynamically adjusting the target perfusion rate in view of the viable cell density, wherein the cells are grown to a high cell density during perfusion.

3. The method of claim 1 or 2, wherein the viable cell density is used in combination with a constant cell-specific perfusion rate to determine the target perfusion rate.

4. The method of claim 3, wherein the constant cell-specific perfusion rate is between about 0.01 to 0.15 nL/cell/day.

5. The method of claim 3 or 4, wherein the constant cell-specific perfusion rate is between about 0.02 to 0.10 nL/cell/day.

6. The method of any one of claims 3-5, wherein the constant cell-specific perfusion rate is about 0.03 nl/cell/day

7. The method of any one of claims 3-5, wherein the constant cell-specific perfusion rate is about 0.04-0.05 nl/cell/day.

8. The method of any one of claim 3-5 or 7, wherein the constant cell-specific perfusion rate is about 0.04 nl/cell/day

9. The method of any one of claims 3-5, wherein the constant cell-specific perfusion rate is between about 0.08 to 0.10 nL/cell/day

10. The method of any one of claim 3-5 or 9, wherein the constant cell-specific perfusion rate is about 0.10 nl/cell/day

11. The method of any one of claims 1-10, wherein the calculation of the target perfusion flow rate is performed by a bioreactor controller.

12. The method of any one of claims 1-11, wherein the perfusion flow rate is increased to achieve the target perfusion flow rate following measurement of the viable cell density in the bioreactor.

13. The method of any one of claims 1-11, wherein the perfusion flow rate is decreased to achieve the target perfusion flow rate following measurement of the viable cell density in the bioreactor.

14. The method of any one of claims 1-13, wherein the viable cell density is further used to measure the time point when a target cell density is reached.

15. The method of claim 14, wherein the target cell density is >30×10⁶ cells/ml, >40 x 10⁶ cells/ml, >50×10⁶ cells/ml, >60×10⁶ cells/ml, or >70×10⁶ cells/ml.

16. The method of claim 14 or 15, wherein the target cell density is >40×10⁶ cell s/ml.

17. The method of any one of claims 1-16, wherein the total perfusate is measured.

18. The method of any one of claims 1-17, wherein the daily perfusion rate is measured.

19. The method of any one of claims 1-18, wherein the capacitance is measured.

20. The method of any one of claims 14-19, wherein the cells are transferred into a production bioreactor upon reaching the target cell density.

21. The method of any one of claims 1-20, wherein the cells produce a polypeptide of interest.

22. The method of claim 21, wherein the polypeptide of interest is an antibody.

23. The method of any one of claims 1-22, wherein the cells are mammalian cells.

24. The method of any one of claims 1-23, wherein the cells are CHO cells.

25. The method of any one of claims 1-24, wherein the perfusion bioreactor is an N-1 perfusion bioreactor.

26. The method of any one of claims 1-25, wherein the perfusion bioreactor is attached to an alternating tangential flow system.

27. The method of any one of claims 1-26, wherein the perfusion bioreactor uses less than about 5, less than about 4.5, less than about 4, less than about 3.5, less than about 3.0, less than about 2.5, less than about 2, less than about 1.5 or less than 1 vessel volumes of perfusion media per day.

28. The method of any one of claims 1-27, wherein the perfusion bioreactor uses only about 25%, about 30%, about 35%, about 40%, about 45%, about 50%, about 55%, about 60%, about 65%, about 70%, about 75%, about 80%, about 85%, about 90%, or about 95% of the total amount of perfusion media used by a process that does not measure the viable cell density.

29. The method of any one of claims 1-28, wherein the biomass capacitance probe is an INCYTE probe.
